(19) United States
(12) Patent Application Publication
(10) Pub. No.: US 2006/0133579 A1
(43) Pub. Date: Jun. 22, 2006

(54) Distortion corrector for digital radiograph

(76) Inventors: Samuel Soonho Lee, Los Angeles, CA (US); Michael Joongkyu Kwon, Los Angeles, CA (US); Grace Heekyong Lee, Los Angeles, CA (US)

Correspondence Address: Eugene Oak, Ph.D., J.D.,;Patent Attorney, 610 S. Van Ness Ave., Los Angeles, CA, 90005, US

(21) Appl. No.: 11/012,992

(22) Filed: Dec. 16, 2004

(51) Int. Cl. A61B 6/14; G03B 42/02; G03C 5/16

(52) U.S. Cl. 378/169

(57) ABSTRACT

A dental X-ray image sensor provided with a cross mark that is opaque to radio waves on the corner of a digital radiography sensor surface renders a computer program to recognize true dimension and to rescale any distortion due to angulational position error in a conventional X-ray digital radiograph system.

### FIELD OF THE INVENTION

The present invention relates generally to a digital radiograph sensor, more specifically to a dental X-ray digital radiograph sensor, marked with a cross that is opaque to x-rays thereon.

### BACKGROUND OF THE INVENTION

Recent development of X-ray digital radiography enables surgical operation of dental diseases with real time observation of the rear inside of a patient's teeth. Direct transport of digital radiographic image into a computer and production of an output image, enlarged to window size on a computer monitor makes it hard to figure out a precise measurement of a tooth or other vital structures. In addition to this, various kinds of distorted of images are produced depending on the position of the radiographic sensor. A distorted image is created due to 1) an angulations and 2) position of the sensor. Angulations of the sensor lead to a radiograph that is expanded or shrunken, laterally or vertically. Such distorted images are seen from bisecting techniques and causes significant error when the clinician is trying to measure the length between critical structures. The purpose of the current invention is to correct angulations errors due to sensor's position, and allow a clinician to precisely measure the distance from point A to point B with a click of a mouse.

### DESCRIPTION OF THE PRIOR ART

U.S. Pat. No. 6,765,609 to Kinoshita illustrates a solid-state image sensor, which has a two-dimensional matrix of a plurality of pixels used to sense the two-dimensional spatial distribution of radioactive rays, light rays, electrons, ions, or the like, is provided with an aperture that extends through a substrate at an image sensing unit on which the pixels are arranged, and a signal transfer path that connects signal transfer electrodes for reading images of the respective pixels kept clear of the aperture.

U.S. Pat. No. 6,693,668 to May, et al. illustrates a self-diagnosing image sensor detects and stores maps of functioning and malfunctioning pixels in a memory directly coupled to the sensor. The memory is coupled to an external monitoring computer, which retrieves the pixel map and adjusts the sensor data received from the image sensor in accordance with the retrieved pixel map. A defect discriminator is coupled directly to the image sensor and to the memory for detecting whether a pixel malfunctions, and updates the map accordingly. U.S. Pat. No. 5,995,583 to Schick, et al. illustrates a method and apparatus for producing a X-ray image of a patient's teeth by placing an intra-oral sensor having a plurality of pixels disposed in a linear array inside the patient's mouth, and moving a radiation source around outside of the patient's mouth. The intra-oral sensor detects the radiation that has passed through the patient's teeth and generates corresponding output signals that depend on the amount of radiation arriving at each pixel. The information contained in the output signals is stored and can be used to create an X-ray image of the patient's teeth. The intra-oral sensor can be moved about inside the patient's mouth in coordination with the movement of the radiation source in order to improve the resulting image quality. This method and apparatus can be used to obtain an X-ray image of any number of teeth, including a panoramic image of all of the patient's teeth, in a single, quick, uninterrupted operation.

U.S. Pat. No. 2,881,655 to Eisenschink and U.S. Pat. No. 5,418,610 to Fischer illustrates a vehicle side mirror and U.S. Pat. No. 4,023,029 to Fischer illustrates a back mirror having an image reflecting surface and a distance indicating light reflective indicia of contrasting reflectivity to the image reflecting surface, the indicia being located in such a position on the mirror that when a just passed vehicle appears on the mirror at a certain location with respect to the indicia, it is safe to pull over into the lane in front of the just passed vehicle. One embodiment of the distance indicating indicia is a is reflective horizontal line located below the center of the mirror. This is just to compare the relative distance between my car and the approaching car from a distorted image on a back mirror, not to measure a real distance from a distorted image.

None of the prior arts illustrates a method of measuring the true distance between two points in real time from distorted images.

### SUMMARY OF THE INVENTION

Recent development of X-ray digital radiography enables surgical operation of dental diseases with real time observation of the rear inside of a patient's teeth. Direct transport of digital radiographic image into a computer and production of an output image, enlarged to window size, on a computer monitor makes it hard to figure out a precise measurement of a tooth or other vital structures. In addition to this, various kind of distorted of images are produced depending on the position of the radiographic sensor. A distorted image is created due to 1) an angulations and 2) position of the sensor. Angulations of the sensor lead to a radiograph that is expanded or shrunken, laterally or vertically. Such a distorted image is seen when using the bisecting technique and causes significant error when the clinician is trying to measure length between critical structures. The purpose of the current invention is to correct angulation's error due to sensor's position, and allow a clinician to precisely measure the distance from point A to point B with a click of a mouse.

A dental X-ray image sensor provided with a radio-opaque cross-mark on the corner of a digital radiograph sensor surface is provided to enable a computer program to recognize true dimension and to rescale any distortion due to angulation's position error in a conventional X-ray digital radiograph system.

### BRIEF DESCRIPTION OF THE DRAWINGS

FIG. 1 is a diagrammatic representation of the intra-oral digital X-ray imaging apparatus of the prior art.

FIG. 2 is a perspective view of a radiography sensor and holder of prior art.

FIG. 3 is a perspective drawing of a radiography sensor engaged inside of a patient's mouth.

FIG. 4 is a schematic drawing showing the distortion of projected image of a tooth depends on the angulations.

FIG. 5 is a schematic drawing of a conventional radiography sensor which additionally has a radio opaque cross mark on a corner of the front surface according to the current application.

FIG. 6-a is a photograph of teeth displayed on a screen of the displayer detected by a radiography sensor, without a radio opaque cross mark, positioned at correct angle.

FIG. 6-b is a photograph of teeth displayed on a screen of the displayer detected by a radiography sensor, without a radio opaque cross mark, positioned laterally angulated.

FIG. 6-c is a photograph of teeth displayed on a screen of the displayer detected by a radiography sensor, without a radio-opaque cross mark, positioned vertically angulated.

FIG. 7-a is a photograph of teeth displayed on a screen of the displayer detected by a radiography sensor, with a radio opaque cross mark, positioned at correct angle.

FIG. 7-b is a photograph of teeth displayed on a screen of the displayer detected by a radiography sensor, with a radio opaque cross mark, positioned laterally angulated.

FIG. 7-c is a photograph of teeth displayed on a screen of the displayer detected by a radiography sensor, without a radio opaque cross mark, positioned vertically angulated.

### DETAILED DESCRIPTION OF THE PRESENTLY PREFERRED EMBODIMENTS

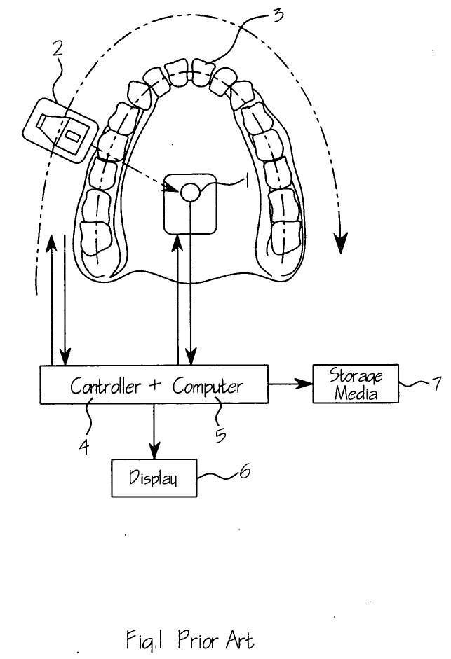

FIG. 1 illustrates a diagrammatic representation of a intra-oral digital X-ray imaging apparatus of the prior art. Most modern x-ray radiographs operate as shown in FIG. 1. A radiography sensor (1) is placed inside of the mouth of a patient. An X-ray source (2) emits X-ray from outside. The X-ray passes through the teeth (3) and projects an image of the teeth on the radiography sensor (1). The X-ray source (2) and the radiography sensor (1) are connected to a controller (4) in which a computer (5) is installed. The image of the teeth (actually X-ray signals disturbed by the teeth) projected on the radiography sensor (1) is fabricated by the computer (5) and projected on a display (6) to monitor the rear inside of the teeth (3) in real time. Selected images are stored in the storage media (7).

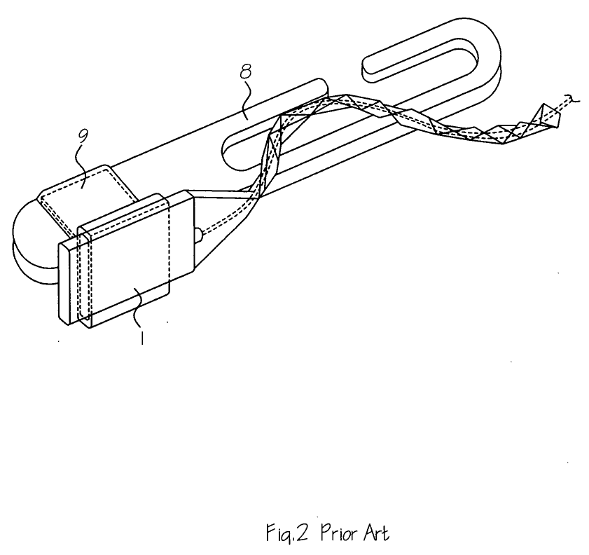

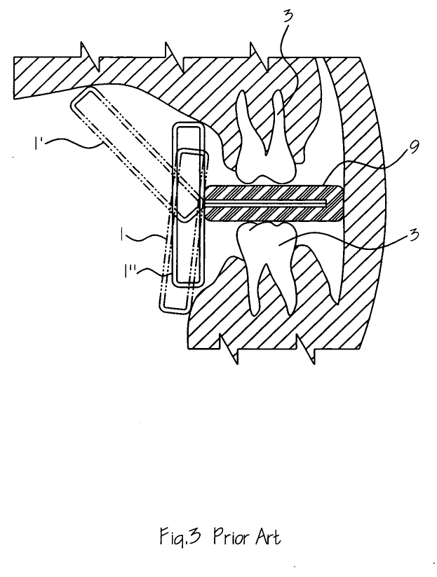

FIG. 2 is a perspective view of a radiography sensor (1) and sensor holder (8) of the prior art. And FIG. 3 is a perspective drawing of a radiography sensor (1) engaged inside a patient's mouth. A patient bites a biting place (9) on the sensor holder (8) with teeth (3). If the patient bites the sensor (1) in upright position, there is no problem. However, if some special place in the mouth should be observed, the radiography sensor (1) should be positioned with an angle to the vertical surface. However, angulations of the sensor can lead to radiograph that is expanded or shrunken, laterally or vertically. This is seen using the bisecting technique. This can contribute to significant error when the clinician is trying to measure length between critical structures.

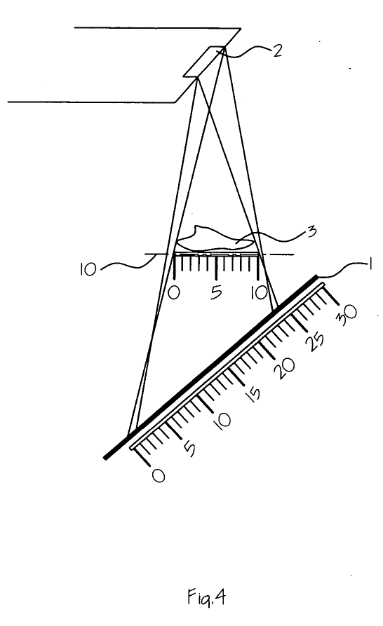

FIG. 4 is a schematic drawing showing the distortion of the projected image of a tooth (3) on the radiography sensor (1) depending on the angulations of the sensor (1) from the parallel plane (10) of the tooth (3). Within the limit of the inside of a human mouth, angulations (11) of 50 degree between the parallel plane (10) and the surface of the radiography sensor (1) leads to extension of the length of the tooth (3) image on the surface of the sensor (1) with a factor over 2.

On the other end, magnification error that occurs due to placing the sensor father away from the object (teeth) is not very significant (˜5%). Because the size of human mouth is not greater than 4 cm (lingual surface of teeth to lingual surface of contra lateral side), and the sensor (1) must be placed with in range of 2 cm from teeth (3) due to shape of oral cavity. This means that enlargement from dental radiograph is not very significant (5% maximum). This data is collected from actual radiographic experiment with penny exposed in different distances. Therefore, the enlargement effect due to the distance from the tooth (3) is ignored when clinician is measuring distances within digital radiograph.

However, the incubational brings significant errors in measuring the real 5 distances between two points on the tooth (3). Therefore, it should be corrected by more accurate and convenient methods than known to experts in the field of his/her endeavor. This will not only allow correction of improper angulations, but also allow user to estimate precise measurements.

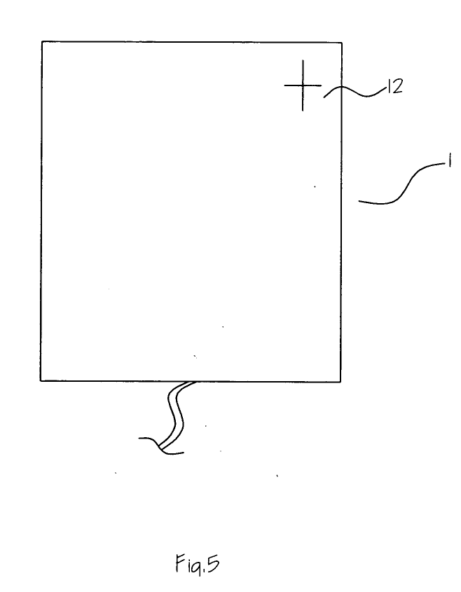

FIG. 5 is a schematic drawing of a conventional radiography sensor (1) which lo additionally has a radio-opaque cross mark (12) on a corner of the front surface according to the current application. The dimension of the cross (12) is predetermined, such as 3 mm by 3 mm. The radio opaque material is, including but not limited to, barium sulfate, lead, steel, and salts.

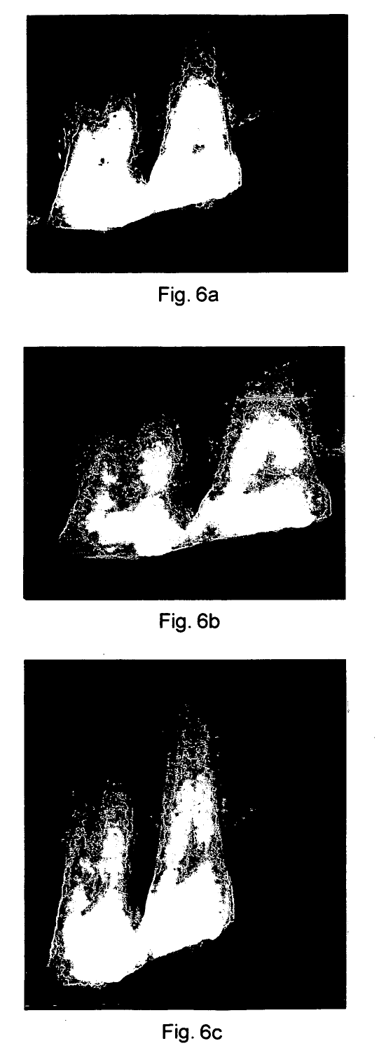

FIG. 6-a is a photograph of teeth (3) displayed on a screen of the displayer (6) is detected by a radiography sensor (1), without a radio opaque cross mark (12), positioned at correct angle.

FIG. 6-b is a photograph of teeth (3) displayed on a screen of the displayer (6) detected by a radiography sensor (1), without a radio-opaque cross mark (12), positioned laterally angulated.

FIG. 6-c is a photograph of teeth (3) displayed on a screen of the displayer (6) detected by a radiography sensor, without a radio-opaque cross mark (12), positioned vertically angulated.

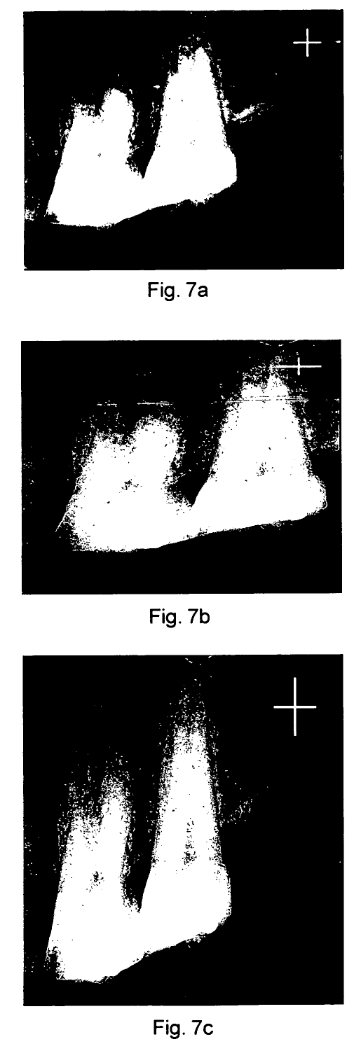

FIG. 7-a is a photograph of teeth (3) displayed on a screen of the displayer (6) detected by a radiography sensor (1), with a radio-opaque cross mark (12), positioned at correct angle.

FIG. 7-b is a photograph of teeth (3) displayed on a screen of the displayer (6) detected by a radiography sensor (1), with a radio-opaque cross mark (12), positioned laterally angulated.

FIG. 7-c is a photograph of teeth (3) displayed on a screen of the displayer (6) detected by a radiography sensor (1), with a radio-opaque cross mark (12), positioned vertically angulated.

It is very hard to determine what is the correct image and how much it is distorted from the FIGS. of 6-a, b, and c. Meanwhile, from FIGS. 7-a, b, and c, a clinician can figure out how much and in what direction the original image is distorted since the dimension of the radio opaque cross (12) is predetermined by the computer (5).

This new simple technology will advance dentistry to the next level, because dentists will be able to more easily and accurately calculate canal length, distance from inferior alveolar nerve, sinus floor, and other critical structures. In return, we believe that patients will benefit from this technology by receiving safer and more precise surgeries.

What is claimed is:

1. A dental X-ray image sensor provided with a radio-opaque cross mark on the corner of a digital radiograph sensor surface renders a computer program to recognize true dimension and to rescale any distortion due to angulational position error in a conventional X-ray digital radiograph system.

2. A dental X-ray image sensor provided with a radio-opaque cross mark of the claim 1, wherein the opaque cross mark is made from barium sulfate.

3. A dental X-ray image sensor provided with a radio-opaque cross mark of claim 1, wherein the length and width of the radio opaque cross mark is 3 mm by 3 mm.
